(19) United States
(12) Patent Application Publication
(10) Pub. No.: US 2013/0274601 A1
(43) Pub. Date: Oct. 17, 2013

(54) ULTRASOUND DIAGNOSIS APPARATUS, IMAGE PROCESSING APPARATUS, AND IMAGE PROCESSING METHOD

(71) Applicants: Toshiba Medical Systems Corporation, Otawara-shi (JP); Kabushiki Kaisha Toshiba, Manato-ku (JP)

(72) Inventors: Mitsuo AKIYAMA, Otawara-shi (JP); Yasuhiko ABE, Otawara-shi (JP)

(21) Appl. No.: 13/911,726

(22) Filed: Jun. 6, 2013

Related U.S. Application Data
(63) Continuation of application No. PCT/JP2011/078597, filed on Dec. 9, 2011.

(30) Foreign Application Priority Data
Dec. 13, 2010 (JP) 2010-277186

(51) Int. Cl. A61B 8/02

(52) U.S. Cl. CPC A61B 8/02
USPC 600/437

(57) ABSTRACT

An ultrasound diagnosis apparatus according to an embodiment includes an input unit and a selecting unit. The input unit receives a group of ultrasound images corresponding to at least one heart beat from among a group of ultrasound images corresponding to a plurality of heart beats of a subject, as first partial data. While using a heart rate of the subject during the acquisition period of the first partial data as a reference heart rate, the selecting unit selects a group of ultrasound images having a heart rate within a predetermined range from the reference heart rate as second partial data, out of a group of ultrasound images of the subject acquired in a time frame different from that of the first partial data.

### CROSS-REFERENCE TO RELATED APPLICATIONS

This application is a continuation of PCT international application Ser. No. PCT/JP2011/078597 filed on Dec. 9, 2011 which designates the United States, and which claims the benefit of priority from Japanese Patent Application No. 2010-277186, filed on Dec. 13, 2010; the entire contents of which are incorporated herein by reference.

### FIELD

Embodiments described herein relate generally to an ultrasound diagnosis apparatus, an image processing apparatus, and an image processing method.

### BACKGROUND

To make a diagnosis of heart diseases, a quantitative evaluation method has conventionally been used by which the cardiac wall motion is quantitatively evaluated by using echo cardiography. Such a quantitative evaluation method using echo cardiography employs an application that quantifies various types of wall motion information by using ultrasound images of the heart corresponding to at least one heart beat. One known example of such an application is configured so as to quantify the cardiac wall motion while using a speckle tracking method by which a point set on a myocardium in ultrasound images is tracked based on a speckle pattern, which is unique to ultrasound images.

Further, while using such an application, an evaluation can be made on changes in a cardiac function, by analyzing a plurality of ultrasound images acquired in mutually-different time frames such as before surgery and after surgery, quantifying the wall motion information, and using the difference in pieces of quantitative information. In a conventional example, when the changes in the cardiac function are quantitatively analyzed between the images acquired in the mutually-different time frames, the difference between the pieces of quantitative information is obtained by matching cardiac phases while using an R-wave as a reference.

However, heart rates, even of healthy people, generally exhibit a physiological fluctuation of less than 10%. Also, many clinical cases of arrhythmia show unstable heart rates as observed in a typical example with atrial fibrillation. Further, during a stress-echo process used for making a diagnosis of ischemic heart diseases, the heart rate is actively made to fluctuate according to the state of a load.

For these reasons, to improve the reliability of a result of a quantitative analysis performed on the changes in the cardiac function, it is ideal to acquire moving-picture data corresponding to a large number of heart beats and to make a comparison by selecting pieces of data having more similar heart rates than others. However, because it is difficult to find the pieces of data having similar heart rates from among the data containing the large number of heart beats, it takes a long time to perform the medical examination.

As explained above, according to the conventional method, when a result is obtained by comparing the pieces of quantitative data with each other while simply using the R-wave as a reference between the images acquired in the mutually-different time frames, the reliability of the result may be low. Accordingly, a burden is imposed on the operator of the apparatus in an attempt to obtain a comparison result having high reliability.

### BRIEF DESCRIPTION OF THE DRAWINGS

FIG. 1 is a drawing for explaining a configuration of an ultrasound diagnosis apparatus according to a first embodiment;

FIG. 2 is a drawing for explaining physiological fluctuations in heart rates;

FIG. 3 is a drawing for explaining first partial data;

FIG. 4, FIG. 5A, FIG. 5B are drawings for explaining a selecting unit according to the first embodiment;

FIG. 6 is a flowchart for explaining a selecting process performed by the ultrasound diagnosis apparatus according to the first embodiment;

FIG. 7 is a flowchart for explaining an analyzing process performed by the ultrasound diagnosis apparatus according to the first embodiment;

FIG. 8 is a drawing for explaining an image processing unit according to a second embodiment;

FIG. 9A and FIG. 9B are drawings for explaining a specifying unit;

FIG. 10 is a flowchart for explaining a selecting process performed by an ultrasound diagnosis apparatus according to the second embodiment;

FIG. 11 is a drawing for explaining a selecting unit according to a third embodiment;

FIG. 12 is a flowchart for explaining a selecting process performed by an ultrasound diagnosis apparatus according to the third embodiment; and

FIG. 13A, FIG. 13B, and FIG. 13C are first drawings for explaining modification examples of the selecting process.

### DETAILED DESCRIPTION

An ultrasound diagnosis apparatus according to an embodiment includes an input unit and a selecting unit. The input unit receives a group of ultrasound images corresponding to at least one heart beat from among a group of ultrasound images corresponding to a plurality of heart beats of a subject, as first partial data. While using a heart rate of the subject during the acquisition period of the first partial data as a reference heart rate, the selecting unit selects a group of ultrasound images having a heart rate within a predetermined range from the reference heart rate as second partial data, out of a group of ultrasound images of the subject acquired in a time frame different from that of the first partial data.

In the following sections, exemplary embodiments of an ultrasound diagnosis apparatus will be explained in detail, with reference to the accompanying drawings.

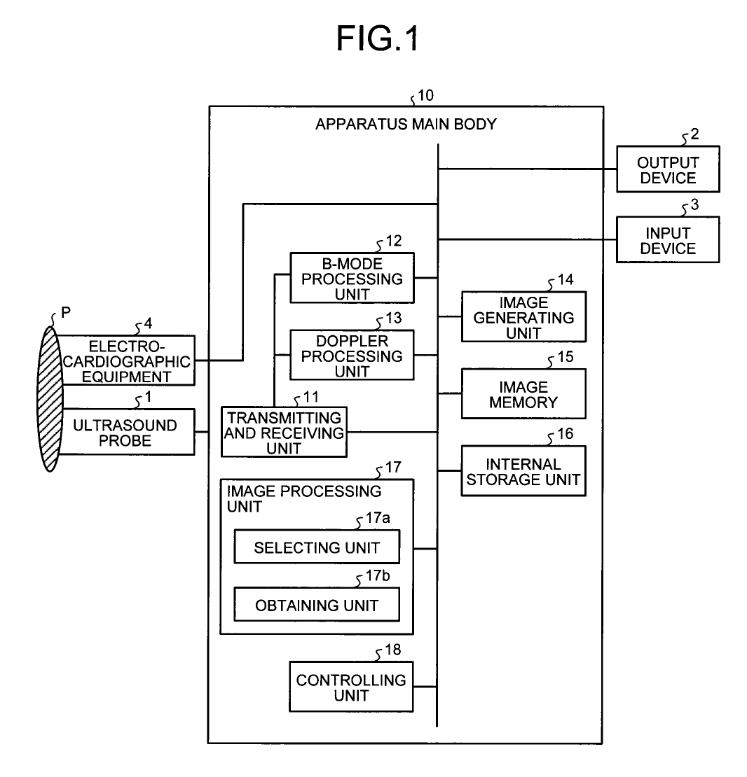

First, a configuration of an ultrasound diagnosis apparatus according to a first embodiment will be explained. FIG. 1 is a drawing for explaining the configuration of the ultrasound diagnosis apparatus according to the first embodiment. As shown in FIG. 1, the ultrasound diagnosis apparatus according to the first embodiment includes an ultrasound probe 1, an output device 2, an input device 3, an electrocardiographic equipment 4, and an apparatus main body 10.

The ultrasound probe 1 is detachably connected to the apparatus main body 10. The ultrasound probe 1 includes a plurality of piezoelectric transducer elements, which generate an ultrasound wave based on a drive signal supplied from a transmitting and receiving unit 11 included in the apparatus main body 10 (explained later). Further, the ultrasound probe 1 receives a reflected wave from an examined subject P and converts the received reflected wave into an electric signal. Further, the ultrasound probe 1 includes matching layers included in the piezoelectric transducer elements, as well as a backing member that prevents ultrasound waves from propagating rearward from the piezoelectric transducer elements.

When an ultrasound wave is transmitted from the ultrasound probe 1 to the subject P, the transmitted ultrasound wave is repeatedly reflected on a surface of discontinuity of acoustic impedances at a tissue in the body of the subject P and is received as a reflected-wave signal by the plurality of piezoelectric transducer elements included in the ultrasound probe 1. The amplitude of the received reflected-wave signal is dependent on the difference between the acoustic impedances on the surface of discontinuity on which the ultrasound wave is reflected. When the transmitted ultrasound pulse is reflected on the surface of a flowing bloodstream or a cardiac wall, the reflected-wave signal is, due to the Doppler effect, subject to a frequency shift, depending on a velocity component of the moving members with respect to the ultrasound wave transmission direction.

It should be noted that the first embodiment is applicable to a situation where the ultrasound probe 1 shown in FIG. 1 is configured with a one-dimensional ultrasound probe in which the plurality of piezoelectric transducer elements are arranged in a row, to a situation where the ultrasound probe 1 is configured with a one-dimensional ultrasound probe in which the plurality of piezoelectric transducer elements arranged in a row are mechanically oscillated, and to a situation where the ultrasound probe 1 is configured with a two-dimensional ultrasound probe in which the plurality of piezoelectric transducer elements are arranged two-dimensionally in a matrix formation. Such a two-dimensional ultrasound probe is able to scan the subject P two-dimensionally, by transmitting ultrasound waves in a converged manner.

The input device 3 includes a mouse, a keyboard, a button, a panel switch, a touch command screen, a foot switch, a trackball, and the like. The input device 3 receives various types of setting requests from an operator of the ultrasound diagnosis apparatus and transfers the received various types of setting requests to the apparatus main body 10.

For example, the input device 3 according to the first embodiment receives a designation to select a group of ultrasound images used for analyzing a cardiac wall motion, from the operator. Further, to analyze the cardiac wall motion, the input device 3 according to the first embodiment receives, from the operator, a designation of the type of quantitative information of the cardiac wall motion calculated from a group of ultrasound images. The specifics of various types of designations received by the input device 3 according to the first embodiment will be explained in detail later.

The output device 2 includes a monitor, a speaker, and the like. The monitor of the output device 2 displays a Graphical User Interface (GUI) used by the operator of the ultrasound diagnosis apparatus to input the various types of setting requests through the input device 3 and displays ultrasound images generated by the apparatus main body 10. Further, the speaker of the output device 2 outputs a sound. For example, the speaker of the output device 2 outputs a predetermined sound such as a beep, for the purpose of informing the operator of a processing status of the apparatus main body 10.

The electrocardiographic equipment 4 is connected to the apparatus main body 10 and is configured to obtain an electrocardiogram (ECG) of the subject P on which an ultrasound scanning process is performed. The electrocardiographic equipment 4 transmits the obtained electrocardiogram to the apparatus main body 10.

The apparatus main body 10 is an apparatus that generates the ultrasound image based on the reflected wave received by the ultrasound probe 1. As shown in FIG. 1, the apparatus main body 10 includes the transmitting and receiving unit 11, a B-mode processing unit 12, a Doppler processing unit 13, an image generating unit 14, an image memory 15, an internal storage unit 16, an image processing unit 17, and a controlling unit 18.

The transmitting and receiving unit 11 includes a trigger generating circuit, a transmission delaying circuit, a pulser circuit, and the like and supplies the drive signal to the ultrasound probe 1. The pulser circuit repeatedly generates a rate pulse for forming a transmission ultrasound wave at a predetermined rate frequency. Further, the transmission delaying circuit applies a transmission delay period that is required to converge the ultrasound wave generated by the ultrasound probe 1 into the form of a beam and to determine transmission directionality and that corresponds to each of the piezoelectric transducer elements, to each of the rate pulses generated by the pulser circuit. Further, the trigger generating circuit applies a drive signal (a drive pulse) to the ultrasound probe 1 with timing based on the rate pulses. In other words, the transmission delaying circuit arbitrarily adjusts the directions of the transmissions from the piezoelectric transducer element surfaces, by varying the transmission delay periods applied to the rate pulses.

The transmitting and receiving unit 11 has a function to be able to instantly change the transmission frequency, the transmission drive voltage, and the like, for the purpose of executing a predetermined scanning sequence based on an instruction from the controlling unit 18 (explained later). In particular, the configuration to change the transmission drive voltage is realized by using a linear-amplifier-type transmitting circuit of which the value can be instantly switched or by using a mechanism configured to electrically switch between a plurality of power source units.

Further, the transmitting and receiving unit 11 includes an amplifier circuit, an Analog/Digital (A/D) converter, a reception delaying circuit, an adder, and the like and generates reflected-wave data by performing various types of processes on the reflected-wave signal received by the ultrasound probe 1. The amplifier circuit amplifies the reflected-wave signal for each of channels and performs a gain correcting process thereon. The A/D converter applies an A/D conversion to the gain-corrected reflected-wave signal. The reception delaying circuit applies a reception delay period required to determine reception directionality, to digital data. The adder generates the reflected-wave data by performing an adding process on reflected-wave signals to which the reception delay periods have been applied by the reception delaying circuit. As a result of the adding process performed by the adder, reflected components from the direction corresponding to the reception directionality of the reflected-wave signal are emphasized.

In this manner, the transmitting and receiving unit 11 controls the transmission directionality and the reception directionality in the transmission and the reception of the ultrasound wave.

The B-mode processing unit 12 receives the reflected-wave data from the transmitting and receiving unit 11 and generates data (B-mode data) in which the strength of each signal is expressed by a degree of brightness, by performing a logarithmic amplification, an envelope detection process, and the like on the received reflected-wave data.

The Doppler processing unit 13 extracts bloodstreams, tissues, and contrast echo components under the influence of the Doppler effect by performing a frequency analysis so as to obtain velocity information from the reflected-wave data received from the transmitting and receiving unit 11, and further generates data (Doppler data) obtained by extracting moving member information such as an average velocity, the dispersion, the power, and the like for a plurality of points.

The image generating unit 14 generates an ultrasound image from the data generated by the B-mode processing unit 12 and the Doppler processing unit 13. More specifically, from the B-mode data generated by the B-mode processing unit 12, the image generating unit 14 generates a B-mode image in which the strength of the reflected wave is expressed by a degree of brightness. Further, from the Doppler data generated by the Doppler processing unit 13, the image generating unit 14 generates an average velocity image, a dispersion image, and a power image, expressing the moving member information, or a color Doppler image, which is an image combining these images.

Further, the image generating unit 14 is also able to generate a synthesized image by synthesizing text information of various parameters, scale graduations, body marks, and the like with an ultrasound image.

In this situation, generally speaking, the image generating unit 14 converts (by performing a scan convert process) a scanning line signal sequence from an ultrasound scan into a scanning line signal sequence in a video format used by, for example, television and generates an ultrasound image serving as a displayed image. Further, as various types of image processing processes other than the scan convert process, the image generating unit 14 performs, for example, an image processing process (a smoothing process) to re-generate a brightness average-value image while using a plurality of image frames resulting from the scan convert process and an image processing process (an edge emphasizing process) by employing a differential filter within an image.

The image memory 15 is a memory for storing therein the ultrasound image generated by the image generating unit 14. In this situation, the image generating unit 14 stores, into the image memory 15, the ultrasound image and the time at which the ultrasound scan was performed to generate the ultrasound image, in correspondence with the electrocardiogram transmitted from the electrocardiographic equipment 4. In other words, by referring to the data stored in the image memory 15, the image processing unit 17 (explained later) is able to obtain a cardiac phase and a heart rate (HR) corresponding to the time when the ultrasound scan was performed to generate the ultrasound image.

Further, in correspondence with the ultrasound image generated by the image generating unit 14, the image memory 15 also stores therein various types of incidental information such as the patient's ID, the examination date, the examination target site, body marks, and the like, in addition to the information about the electrocardiogram. Further, the image memory 15 is also able to store therein data generated by the B-mode processing unit 12 and the Doppler processing unit 13. The data generated by the B-mode processing unit 12 and the Doppler processing unit 13 may be referred to as raw data.

The internal storage unit 16 stores therein various types of data such as a control computer program (hereinafter, “control program”) to realize ultrasound transmissions and receptions, image processing, and display processing, as well as diagnosis information (e.g., patients' IDs, medical doctors' observations), diagnosis protocols, and various types of body marks. Further, the internal storage unit 16 may be used, as necessary, for storing therein any of the images stored in the image memory 15. Furthermore, the data stored in the internal storage unit 16 can be transferred to any external peripheral device via an interface circuit (not shown).

The image processing unit 17 is a processing unit that performs various types of image processing processes on the ultrasound images stored in the image memory 15. As shown in FIG. 1, the image processing unit 17 includes a selecting unit 17a and an obtaining unit 17b. The processes performed by the image processing unit 17 according to the first embodiment will be explained in detail later.

The controlling unit 18 controls the entire processes performed by the ultrasound diagnosis apparatus. More specifically, based on the various types of setting requests input by the operator via the input device 3 and various types of control programs and various types of data read from the internal storage unit 16, the controlling unit 18 controls processes performed by the transmitting and receiving unit 11, the B-mode processing unit 12, the Doppler processing unit 13, the image generating unit 14, and the image processing unit 17. Further, the controlling unit 18 exercises control so that the monitor of the output device 2 displays the ultrasound images stored in the image memory 15, a GUI used for designating various types of processes performed by the image processing unit 17, and processing results of the image processing unit 17. Further, the controlling unit 18 exercises control so that the speaker of the output device 2 outputs the predetermined sound, based on the processing results of the image processing unit 17.

An overall configuration of the ultrasound diagnosis apparatus according to the first embodiment has thus been explained. By using the ultrasound diagnosis apparatus according to the first embodiment configured as described above, the operator makes an evaluation related to changes in a cardiac function, between groups of ultrasound images acquired in mutually-different time frames, such as before surgery and after surgery.

As mentioned earlier, however, heart rates (HR), even of healthy people, generally exhibit a physiological fluctuation of less than 10%. Also, many clinical cases of arrhythmia show unstable heart rates as observed in a typical example with atrial fibrillation. Further, during a stress-echo process used for making a diagnosis of ischemic heart diseases, the heart rate is actively made to fluctuate according to the state of a load.

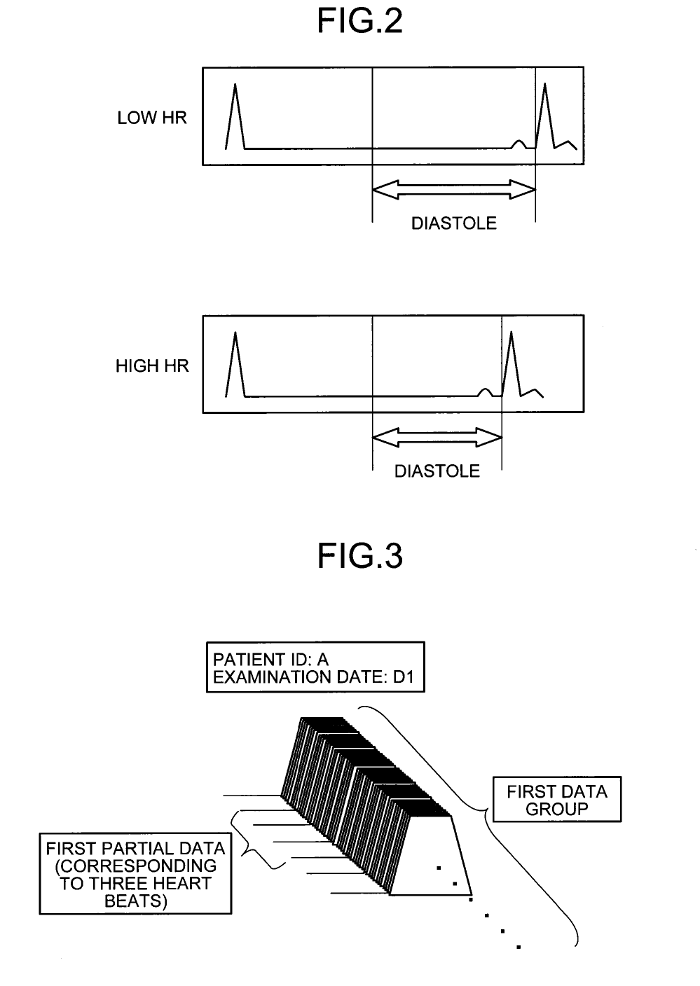

In this situation, when the heart rates are different between the groups of images used as comparison targets, difference information over the course of time (hereinafter, “chronological difference information”) of quantitative information of the cardiac wall motion calculated from the groups of images may be, in some situations, different from a result that is originally expected. This difference is caused by a clinical phenomenon where, even if the heart rates are different due to the physiological fluctuation, the duration of the systole does not change very much, whereas the duration of the diastole (the time period from an early diastole to an atrial systole) dominantly changes. FIG. 2 is a drawing for explaining the physiological fluctuations in heart rates.

More specifically, with reference to FIG. 2, let us compare a situation where the heart rate (HR) is low with a situation where the heart rate (HR) is high. Although the durations of the systole are substantially equal, the duration of the diastole with a low HR is longer than the duration of the diastole with a high HR. When the heart rate has such a physiological fluctuation, if pieces of quantitative data are obtained while using the R-wave as a reference, the durations of the atrial systole will be different from each other. For example, according to a conventional method that is generally used, while the time interval of an R-wave (an R-R interval) is taken as 100%, an elapsed time period since the R-wave is converted to a value relative to the R-R interval, so that groups of ultrasound images each corresponding to one heart beat and having been acquired in mutually-different time frames are compared with each other. According to this method, however, if the heart rates are different between the groups of images, the chronological difference information is not represented by data reflecting a comparison of data from mutually the same cardiac phase.

Further, when a stress-echo process is performed, because the heart rate is actively made to fluctuate, the fluctuation in the heart rate is generally larger than a physiological fluctuation, even in the same cardiac phase. In that situation, it is ideal to acquire moving-picture data corresponding to a large number of heart beats and to make a comparison by selecting pieces of data having more similar heart rates than others. However, because it is difficult to find the pieces of data having similar heart rates from among the data containing the large number of heart beats, it takes a long time to perform the medical examination. The same problem occurs in situations with arrhythmia, such as those with atrial fibrillation, as described above.

As explained above, according to the conventional method, when a result is obtained by comparing the pieces of quantitative information with each other while simply using the R-wave as a reference between the images acquired in the mutually-different time frames, the reliability of the result may be low. Accordingly, a burden is imposed on the operator in an attempt to obtain a comparison result having high reliability.

To cope with this situation, the ultrasound diagnosis apparatus according to the first embodiment performs a process by employing the image processing unit 17 shown in FIG. 1, to easily improve the reliability of the quantitative analysis performed on the changes in the cardiac function between the mutually-different time frames.

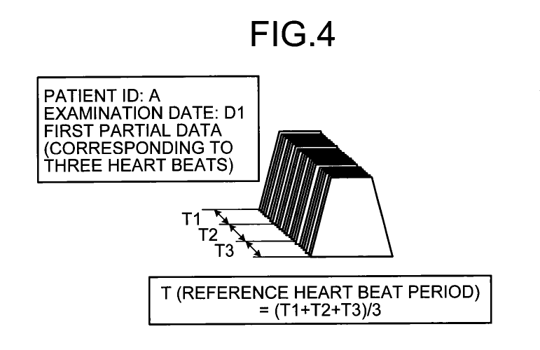

First, the input device 3 receives a group of ultrasound images corresponding to at least one heart beat from among a group of ultrasound images corresponding to a plurality of heart beats of the subject P, as first partial data. In this situation, the group of ultrasound images out of which the first partial data is selected will be referred to as a first data group. Let us assume that the first partial data is selected by the operator. In that situation, the input device 3 receives, as the first partial data, the group of ultrasound images corresponding to at least one heart beat selected by the operator, out of the first data group containing the group of ultrasound images corresponding to the plurality of heart beats of the subject P. FIG. 3 is a drawing for explaining the first partial data.

For example, as shown in FIG. 3, the operator inputs, via the input device 3, “patient ID: A” of the subject P and “examination date: D1” to be used as the targets of an analysis performed on the changes in the cardiac function. As a result, the controlling unit 18 obtains, out of the image memory 15, the first data group containing the group of ultrasound images corresponding to the plurality of heart beats of the subject P acquired on the “examination date: D1”. Subsequently, the controlling unit 18 causes, for example, the monitor of the output device 2 to display the obtained first data group in the form of a moving picture or thumbnails. After that, by referring to the monitor, the operator selects the group of ultrasound images corresponding to a single heart beat or a plurality of heart beats, as the first partial data, which is represented by one of the groups of images used for analyzing the changes in the cardiac function. In this situation, together with the ultrasound images displayed on the monitor, the heart rates obtained from the electrocardiographic equipment 4 are displayed at the same time.

Further, as shown in FIG. 3 for example, the operator selects a group of ultrasound images corresponding to three consecutive heart beats as the first partial data. Information about the first partial data received by the input device 3 is provided for the selecting unit 17a included in the image processing unit 17 shown in FIG. 1, via the controlling unit 18.

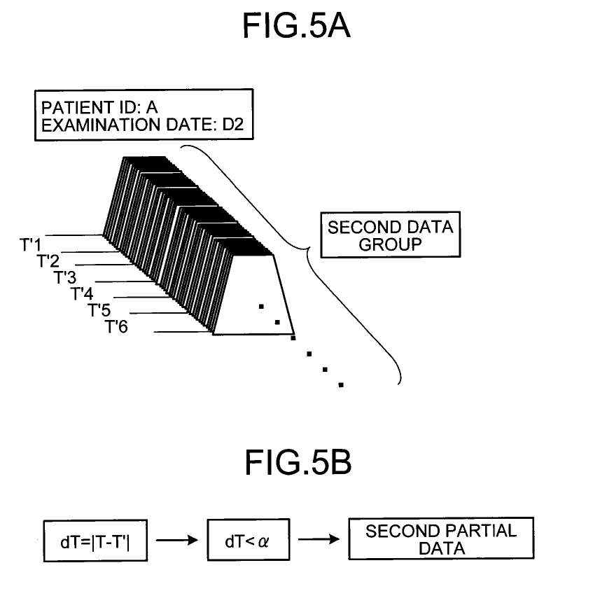

The selecting unit 17a shown in FIG. 1 determines the heart rate of the subject P during the acquisition period of the first partial data received by the input device 3 to be a reference heart rate. In this situation, according to the first embodiment, the selecting unit 17a calculates a reference heart beat period, based on the reference heart rate. A heart rate is, for example, the number of times the heart beats per second. A heart beat period is expressed by, for example, the reciprocal number of the heart rate. In other words, a heart beat period denotes the period of time it takes for the heart to beat once. The heart beat period corresponds to, for example, an R-R interval. When the first partial data corresponds to a plurality of heart beats, the selecting unit 17a calculates an average value of the heart beat periods of the heart beats, as the reference heart beat period. FIGS. 4, 5A, and 5B are drawings for explaining the selecting unit according to the first embodiment.

For example, the selecting unit 17a obtains heart beat periods (T1, T2, T3) of the heart beats, by using an electrocardiogram kept in correspondence with the first partial data (corresponding to the three heart beats) identified with “patient ID: A; examination date: D1”, out of the image memory 15. Further, the selecting unit 17a determines the average value “(T1+T2+T3)/3” of “T1, T2, and T3” to be a reference heart beat period “T”. In this situation, if the first partial data corresponds to a single heart beat, the selecting unit 17a simply determines the heart rate of the first partial data to be the reference heart rate and calculates the reference heart beat period. Further, the reference heart beat period calculated based on the first partial data corresponding to a plurality of heart beats may be expressed by using any representative value calculated statistically (e.g., a median), other than the average value.

After that, the selecting unit 17a selects, as second partial data, a group of ultrasound images having a heart rate within a predetermined range from the reference heart rate, out of a group of ultrasound images of the subject P acquired in a time frame different from that of the first partial data. In this situation, the group of ultrasound images out of which the second partial data is selected will be referred to as a second data group. Also, in the first embodiment, let us assume that the reference heart beat period corresponding to the reference heart rate is used. In that situation, the selecting unit 17a selects, as the second partial data serving as a comparison target to be compared with the first partial data, the group of ultrasound images having a heart beat period within a predetermined range from the reference heart beat period, out of the second data group represented by the group of ultrasound images of the subject P acquired in the time frame different from that of the first data group. It is also acceptable to configure the first embodiment in such a manner that the second partial data is selected by using the reference heart rate.

In this situation, the selecting unit 17a according to the first embodiment performs the selecting process while using the group of ultrasound images of the subject P already acquired in the time frame different from that of the first data group, as the second data group. In other words, the operator selects, out of the pieces of data stored in the image memory 15, the second data group from which the second partial data, which serves as the comparison target to be compared with the first partial data, is selected.

For example, as shown in FIG. 5A, to designate the second data group, the operator inputs, via the input device 3, “patient ID: A” of the subject P and “examination date: D2”. In this situation, the examination date “D2” may be later than the examination date “D1” or may be earlier than the examination date “D1”. Also, the examination date does not necessarily have to be one. A plurality of examination dates other than D1 may be selected.

Further, as shown in FIG. 5A, for example, the selecting unit 17a performs the process to select the second partial data, while using the entirety of the group of ultrasound images kept in correspondence with “patient ID: A” and “examination date: D2” as the second data group.

It is also acceptable to configure the first embodiment in such a manner that, as explained below, a group of images selected by the operator out of the group of ultrasound images kept in correspondence with “patient ID: A” and “examination date: D2” is determined to be the second data group. When the process to select the second data group is performed, the controlling unit 18 obtains the second data group of the subject P acquired on the “examination date: D2” out of the image memory 15, according to, for example, a display request from the operator. Further, the controlling unit 18 exercises control so that, for example, the monitor of the output device 2 displays the obtained second data group in the form of a moving picture or thumbnails. After that, by referring to the monitor, the operator selects a group of ultrasound images corresponding to a single heart beat or a plurality of heart beats, as the second data group. For example, the operator selects a group of ultrasound images corresponding to six consecutive heart beats shown in FIG. 5A, as the second data group.

After that, the selecting unit 17a divides the second data group into groups of ultrasound images each corresponding to one heart beat and performs a selecting process, while using the groups of ultrasound images resulting from the division as search target groups.

More specifically, the selecting unit 17a performs the selecting process while using a predetermined threshold value “α” that is set in advance. For example, while using the value “α”, the selecting unit 17a sequentially compares heart beat periods “T′1, T′2, T′3, T′4, T′5, T′6, . . . ” in the acquisition periods of the search target groups shown in FIG. 5A, with the reference heart beat period “T”, starting with the search target group at the head.

In other words, as shown in FIG. 5B, the selecting unit 17a calculates the absolute value “dT” of the difference between the reference heart beat period “T” and the search target group heart beat period “T′”. The selecting unit 17a then compares “dT” with “α”. In this situation, if “dT<α” is satisfied, the selecting unit 17a determines that the heart beat period of the search target group is substantially equal to the reference heart beat period and selects the search target group as second partial data. On the contrary, if “dT” is equal to or larger than “α”, the selecting unit 17a determines that the heart beat period of the search target group is not equal to the reference heart beat period and does not select the search target group as second partial data.

As a result of the selecting process performed by the selecting unit 17a, the operator is able to proceed to an analyzing process that follows, without the need to perform the operation to search for pieces of data having similar heart rates from among the data containing a large number of heart beats. In this situation, it is acceptable to configure the selecting unit 17a so as to select a group of ultrasound images of which the acquisition period is substantially equal to the acquisition period of the first partial data, as the second partial data. In that situation, for example, if the first partial data corresponds to three consecutive heart beats, the selecting unit 17a selects a group of ultrasound images corresponding to three consecutive heart beats as the second partial data.

However, even if the second partial data having a heart rate substantially equal to that of the first partial data is automatically selected, the difference information may be, in some situations, different from a result that is originally expected, if “the image quality and/or the observed location” are different between the pieces of partial data.

This phenomenon occurs frequently during an image taking process using an ultrasound diagnosis apparatus. A cause of this phenomenon lies in the fact that, when using an ultrasound diagnosis apparatus, it is difficult to adjust the position of the ultrasound probe 1 or the like. More specifically, the orientation and the position of the ultrasound probe 1 rendering the heart may be different for different patients, and the patient may be hurt depending on the posture of the patient. Thus, during an image acquiring process, the position of the ultrasound probe 1 and/or the posture of the patient are adjusted many times.

To cope with this situation, according to the first embodiment, the following process is performed, after the second partial data is selected: The controlling unit 18 exercises control so that the monitor of the output device 2 displays the group of ultrasound images selected as the second partial data by the selecting unit 17a as second candidate partial data. Further, if the operator who has referred to the second candidate partial data displayed on the monitor under the control of the controlling unit 18 determines the second candidate partial data as a comparison target to be compared with the first partial data, the selecting unit 17a confirms the second candidate partial data to be second partial data.

In other words, by referring to the monitor, the operator judges whether the image quality and the observed location of the second candidate partial data are the same as those of the first partial data. For example, let us assume that the first partial data represents a group of images that are acquired for the purpose of performing an examination on the heart of the subject P and are obtained by taking images of the apical four chambers in the heart of the subject P. In that situation, if the second candidate partial data represents a group of images acquired for the purpose of performing an examination on the liver of the subject P, the operator determines that the second candidate partial data will not be used as second partial data, because the image taking site is different.

Further, if the second candidate partial data represents a group of images that are obtained by taking images of the apical two chambers in the heart, long-axis images of the left margin of the ribs, or short-axis images of the left margin of the ribs of the subject P, the operator determines that the second candidate partial data will not be used as second partial data because the image taking cross-section is different. As another example, even if the second candidate partial data represents a group of images that are obtained by taking images of the apical four chambers in the heart of the subject P, the operator determines that the second candidate partial data will not be used as second partial data if, for example, the operator determines that the image quality (e.g., a contrast value) is different.

On the contrary, if the image taking site, the image taking cross-section, and the image quality of the second candidate partial data are the same as the image taking site, the image taking cross-section, and the image quality of the first partial data, the operator presses, for example, a “confirm button” included in the input device 3. As a result, the selecting unit 17a confirms the second candidate partial data, which has been determined by the operator as the comparison target to be compared with the first partial data, to be the second partial data. For example, within the image memory 15, the selecting unit 17a appends a flag serving as incidental information indicating “second partial data”, to the group of ultrasound images confirmed to be the second partial data.

Returning to the description of FIG. 1, by using the first partial data and the second partial data, the obtaining unit 17b included in the image processing unit 17 obtains chronological difference information between the pieces of partial data. More specifically, the obtaining unit 17b calculates quantitative information quantifying cardiac wall motion information in a region of interest of the subject P, based on the first partial data and the second partial data. The obtaining unit 17b further obtains the chronological difference between the pieces of quantitative information calculated based on the first partial data and the second partial data. Further, the controlling unit 18 exercises control so that the monitor of the output device 2 displays the difference information obtained by the obtaining unit 17b.

Even more specifically, the obtaining unit 17b calculates the quantitative information quantifying the cardiac wall motion information in the region of interest designated by the operator, by tracking a track point that is set on the myocardial tissue rendered in the pieces of partial data, based on a speckle pattern. For example, the obtaining unit 17b calculates, for each of the cardiac phases, a luminal volume of the heart rendered in each of the pieces of partial data.

In another example, the obtaining unit 17b calculates, for each of the cardiac phases, a strain of a myocardial tissue (a myocardial strain), a strain rate of a myocardial tissue (a myocardial strain rate), a displacement of a myocardial tissue (a myocardial displacement), or a displacement speed of a myocardial tissue (a myocardial speed). For example, the obtaining unit 17b may calculate a luminal volume, a myocardial strain, a myocardial strain rate, or a myocardial speed of the entire myocardial tissue for each of the cardiac phases. In yet another example, the obtaining unit 17b may calculate a luminal volume, a myocardial strain, a myocardial strain rate, or a myocardial speed of a local myocardial tissue (e.g., a ventricle) for each of the cardiac phases. If the first partial data and the second partial data each correspond to a plurality of heart beats, the obtaining unit 17b is also able to calculate an average value of luminal volume values, myocardial strain values, myocardial strain rates, or myocardial speeds, for each of the cardiac phases.

In yet another example, if the first partial data and the second partial data are represented by data groups each corresponding to a plurality of heart beats, the obtaining unit 17b calculates, as the quantitative information of the cardiac wall motion corresponding to the heart beats, a myocardial volume, as well as a myocardial mass by multiplying the myocardial volume by an average myocardial density value. Further, the obtaining unit 17b calculates a mass-index value by normalizing the myocardial mass by using a body surface area.

Further, the obtaining unit 17b calculates a difference value between the quantitative information of the cardiac wall motion from the first partial data and the quantitative information of the cardiac wall motion from the second partial data, as the difference information. In another example, as the difference information, the obtaining unit 17b generates a table, a chart, an image, or the like, so that the operator is able to easily compare the pieces of quantitative information of the cardiac wall motion from the first partial data and the second partial data. For example, the obtaining unit 17b generates a chart by plotting the luminal volume for each of the cardiac phases from the first partial data and the second partial data.

In yet another example, the obtaining unit 17b generates a plurality of distribution images in a time sequence each of which is obtained by converting a local myocardial strain corresponding to each of the cardiac phases from the first partial data and the second partial data to a color based on a Look-Up Table (LUT) that is set in advance and mapping the conversion result in, for example, a polar map.

Further, under the control of the controlling unit 18, the monitor of the output device 2 displays the difference information obtained by the obtaining unit 17b.

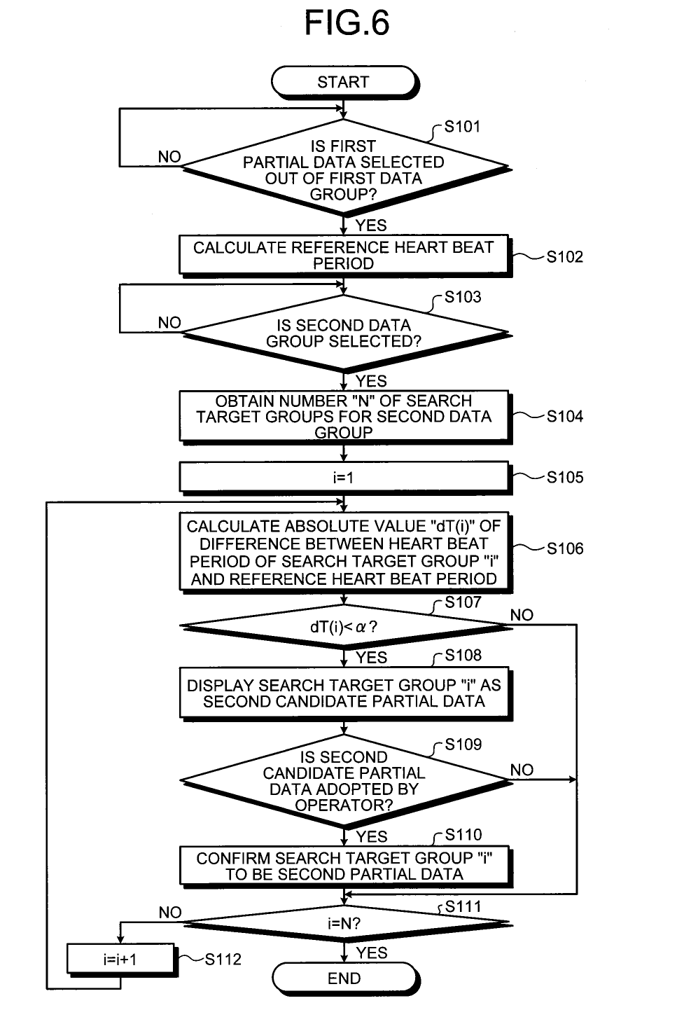

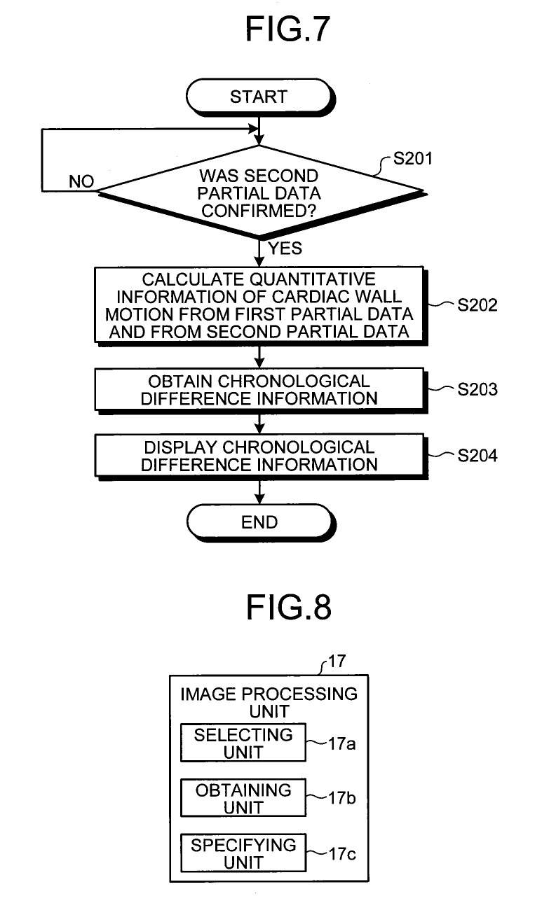

Next, a process performed by the ultrasound diagnosis apparatus according to the first embodiment will be explained, with reference to FIGS. 6 and 7. FIG. 6 is a flowchart for explaining the selecting process performed by the ultrasound diagnosis apparatus according to the first embodiment. FIG. 7 is a flowchart for explaining the analyzing process performed by the ultrasound diagnosis apparatus according to the first embodiment.

As shown in FIG. 6, the ultrasound diagnosis apparatus according to the first embodiment judges whether first partial data is selected out of a first data group (step S101). In other words, the controlling unit 18 judges whether the operator has selected the first partial data via the input device 3. In this situation, if no first partial data is selected (step S101: No), the ultrasound diagnosis apparatus according to the first embodiment goes into a standby state.

On the contrary, if first partial data is selected (step S101: Yes), the selecting unit 17a, which is notified by the controlling unit 18 that the first partial data is selected, calculates a reference heart beat period (T) (step S102). Further, the selecting unit 17a judges whether a second data group is selected (step S103). In this situation, if no second data group is selected (step S103: No), the selecting unit 17a goes into a standby state.

On the contrary, if a second data group is selected (step S103: Yes), the selecting unit 17a obtains the number “N” of search target groups for the second data group (step S104). In other words, the selecting unit 17a obtains the value of “N” indicating how many heart beats the second data group correspond to. After that, the selecting unit 17a sets “i=1” (step S105) and calculates the absolute value “dT(i)” of the difference between the heart beat period of a search target group “i” and the reference heart beat period (step S106).

Subsequently, the selecting unit 17a judges whether “dT(i)” is smaller than a threshold value “α” that is set in advance (step S107). In this situation, if “dT(i)” is smaller than the threshold value “α” (step S107: Yes), the monitor of the output device 2 displays the search target group “i” as second candidate partial data, under the control of the controlling unit 18 (step S108). After that, the selecting unit 17a judges whether the operator adopted the search target group “i” as second partial data (step S109).

In this situation, if the operator adopted the search target group “i” (step S109: Yes), the selecting unit 17a confirms the search target group “i” to be the second partial data (step S110).

In this situation, after the process at step S110 is performed, or if “dT(i)” is equal to or larger than the threshold value “α” (step S107: No), or if the operator did not adopt the search target group “i” (step S109: No), the selecting unit 17a judges whether “i=N” is satisfied (step S111). In this situation, if “i” is smaller than “N” (step S111: No), the selecting unit 17a sets “i=i+1” (step S112) and returns to step S106 where the selecting unit 17a calculates the absolute value “dT(i)” of the difference between the heart beat period of the next search target group “i” and the reference heart beat period.

On the contrary, if “i=N” is satisfied (step S111: Yes), the ultrasound diagnosis apparatus according to the first embodiment ends the selecting process.

After that, as shown in FIG. 7, the ultrasound diagnosis apparatus according to the first embodiment judges whether second partial data was confirmed (step S201). In this situation, if the second partial data was not confirmed (step S201: No), the ultrasound diagnosis apparatus according to the first embodiment goes into a standby state.

On the contrary, if the second partial data was confirmed (step S201: Yes), the obtaining unit 17b calculates quantitative information of the cardiac wall motion from the first partial data and from the second partial data (step S202).

After that, the obtaining unit 17b obtains chronological difference information between the pieces of quantitative information of the cardiac wall motion from the first partial data and from the second partial data (step S203). After that, under the control of the controlling unit 18, the monitor of the output device 2 displays the chronological difference information (step S204), and the process ends.

As explained above, in the first embodiment, the input device 3 receives, as the first partial data, the group of ultrasound images corresponding to at least one heart beat and having been selected by the operator from among the first data group containing the group of ultrasound images corresponding to the plurality of heart beats of the subject P. After that, the selecting unit 17a determines the heart rate of the subject P during the acquisition period of the first partial data received by the input device 3, to be the reference heart rate. Subsequently, the selecting unit 17a selects the group of ultrasound images having the heart rate within the predetermined range from the reference heart beat period, out of the second data group represented by the group of ultrasound images of the subject P acquired in the time frame different from that of the first data group, as the second partial data, which serves as the comparison target to be compared with the first partial data. More specifically, in the first embodiment, the selecting unit 17a performs the selecting process by using the group of ultrasound images of the subject P already acquired in the time frame different from that of the first data group, as the second data group. In the first embodiment, the selecting unit 17a selects the second partial data by comparing the reference heart beat period calculated from the reference heart rate, with the heart beat period calculated from the heart rate of the second data group out of which the second partial data is selected.

In other words, according to the first embodiment, it is possible to automatically select the second partial data having a heart beat period substantially equal to the heart beat period of the first partial data, out of the second data group obtained at the different time from that of the first partial data. With this arrangement, the operator is able to proceed to the process of analyzing the cardiac function performed by using the pieces of data having the similar heart rates, without the need to perform the operation to search for pieces of data having similar heart rates from among the data containing a large number of heart beats. Consequently, according to the first embodiment, it is possible to easily improve the reliability of the quantitative analysis performed on the changes in the cardiac function between the mutually-different time frames.

In addition, in the first embodiment, the controlling unit 18 exercises control so that the monitor of the output device 2 displays the group of ultrasound images selected as the second partial data by the selecting unit 17a, as the second candidate partial data. After that, if the operator who refers to the second candidate partial data displayed on the monitor under the control of the controlling unit 18 determines the second candidate partial data as the comparison target to be compared with the first partial data, the selecting unit 17a confirms the second candidate partial data to be the second partial data.

In other words, according to the first embodiment, the operator selects the group of ultrasound images having the same “image quality and observed location” as those of the first partial data, out of the second candidate partial data, so that it is possible to confirm the second candidate partial data selected by the operator to be the second partial data. Consequently, according to the first embodiment, it is possible to improve, with certainty, the reliability of the quantitative analysis performed on the changes in the cardiac function between the mutually-different time frames.

Furthermore, in the first embodiment, the obtaining unit 17b obtains the chronological difference information between the pieces of partial data, by using the first partial data and the second partial data. More specifically, the obtaining unit 17b calculates the quantitative information quantifying the cardiac wall motion information in the region of interest of the subject P, based on the first partial data and the second partial data, and obtains the chronological difference between the pieces of quantitative information calculated from the pieces of partial data, as the difference information. After that, the controlling unit 18 exercises control so that the monitor of the output device 2 displays the difference information. In other words, according to the first embodiment, it is possible to perform the quantitative analysis on the changes in the cardiac function between the mutually-different time frames and the analysis result outputting process, within the same apparatus where the second partial data was selected.

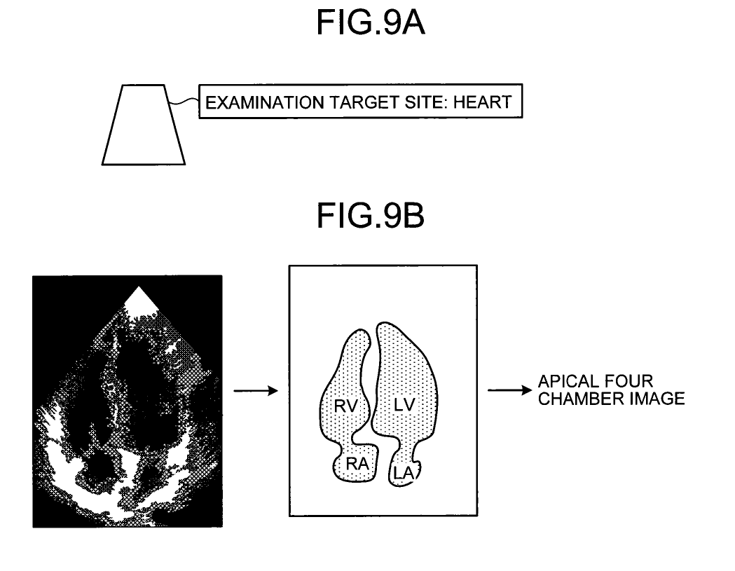

As a second embodiment, an example in which second partial data is automatically confirmed will be explained, with reference to FIG. 8 and so on. FIG. 8 is a drawing for explaining an image processing unit according to the second embodiment.

As shown in FIG. 8, the image processing unit 17 according to the second embodiment is different from the image processing unit 17 according to the first embodiment shown in FIG. 1, for further including a specifying unit 17c. The second embodiment will be explained below while a focus is placed on this difference. In the second embodiment also, the selecting unit 17a selects the second partial data having a heart rate substantially equal to that of the first partial data, out of the already-acquired second data group, like in the first embodiment. The second embodiment is also applicable to a situation where the selecting unit 17a selects a group of ultrasound images of which the acquisition period is substantially equal to the acquisition period of the first partial data, as the second partial data. Further, in the second embodiment also, the selecting process may be performed by using the heart rate or may be performed by using the heart beat period.

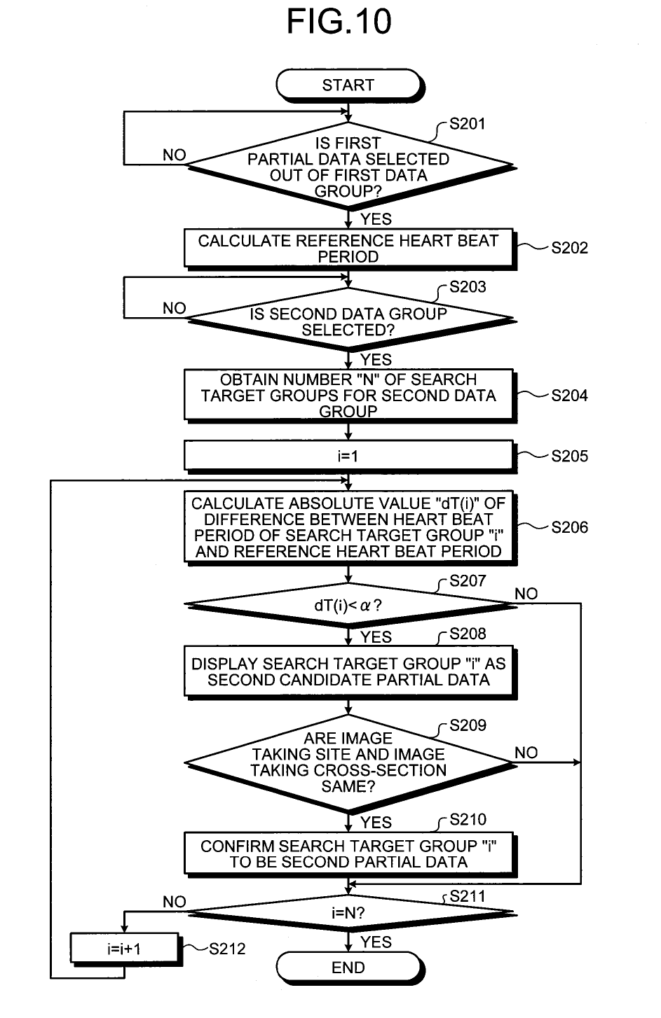

After that, the specifying unit 17c specifies a group of ultrasound images having the same image taking site and the same image taking cross-section as those of the first partial data, out of the second partial data. Further, the selecting unit 17a according to the second embodiment confirms the group of ultrasound images specified by the specifying unit 17c to be second partial data. FIGS. 9A and 9B are drawings for explaining the specifying unit.

For example, the specifying unit 17c refers to the examination target site indicated by the incidental information appended to the ultrasound images. Further, as shown in FIG. 9A, the specifying unit 17c specifies a group of ultrasound images to which “examination target site: heart” is appended out of the group of ultrasound images selected as the second partial data, as the group of ultrasound images having the same image taking site as that of the first partial data.

Further, the specifying unit 17c specifies a group of images having the same image taking cross-section as that of the first partial data, out of the group of ultrasound images having the same image taking site as that of the first partial data. More specifically, the specifying unit 17c specifies a group of ultrasound images having substantially the same characteristic amount as that of the first partial data as the group of ultrasound images having the same image taking cross-section as that of the first partial data, by analyzing the characteristic amounts of the ultrasound images by performing an image processing process on the group of ultrasound images having the same image taking site as that of the first partial data. For example, if the first partial data represents images of the apical four chambers, the specifying unit 17c generates, as shown in FIG. 9B, a binary image by applying threshold value processing to the ultrasound images of which the image taking site is the heart. Further, as shown in FIG. 9B, the specifying unit 17c obtains the four chambers as characteristic amounts, by analyzing the binary image. After that, as shown in FIG. 9B, the specifying unit 17c determines the ultrasound image from which the four chambers were obtained as the characteristic amounts, to be an image of the apical four chambers in which the right atrium (RA), the right ventricle (RV), the left atrium (LA), and the left ventricle (LV) are rendered.

Further, when the group of ultrasound images having the same image taking site and the same image taking cross-section as those of the first partial data have been specified by the specifying unit 17c out of the second partial data, the obtaining unit 17b according to the second embodiment determines the specified group of ultrasound images to be a target from which difference information is obtained.

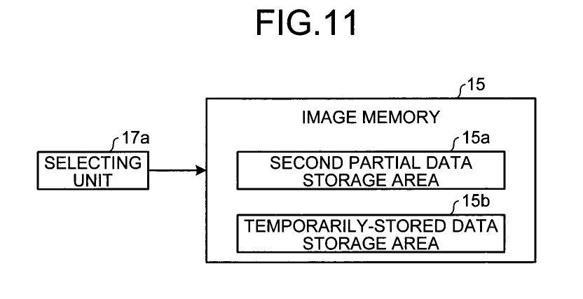

Next, a process performed by the ultrasound diagnosis apparatus according to the second embodiment will be explained, with reference to FIG. 10. FIG. 10 is a flowchart for explaining a selecting process performed by the ultrasound diagnosis apparatus according to the second embodiment. The analyzing process performed by the ultrasound diagnosis apparatus according to the second embodiment is the same as the analyzing process performed by the ultrasound diagnosis apparatus according to the first embodiment explained with reference to FIG. 7. The explanation thereof will be therefore omitted.

As shown in FIG. 10, the ultrasound diagnosis apparatus according to the second embodiment judges whether first partial data is selected out of a first data group (step S201). In this situation, if no first partial data is selected (step S201: No), the ultrasound diagnosis apparatus according to the second embodiment goes into a standby state.

On the contrary, if first partial data is selected (step S201: Yes), the selecting unit 17a calculates a reference heart beat period (T) (step S202). Further, the selecting unit 17a judges whether a second data group is selected (step S203). In this situation, if no second data group is selected (step S203: No), the selecting unit 17a goes into a standby state.

On the contrary, if second partial data group is selected (step S203: Yes), the selecting unit 17a obtains the number “N” of search target groups for the second data group (step S204). After that, the selecting unit 17a sets “i=1” (step S205) and calculates the absolute value “dT(i)” of the difference between the heart beat period of the search target group “i” and the reference heart beat period (step S206).

Subsequently, the selecting unit 17a judges whether “dT(i)” is smaller than a threshold value “α” that is set in advance (step S207). In this situation, if “dT(i)” is smaller than the threshold value “α” (step S207: Yes), the monitor of the output device 2 displays the search target group “i” as second candidate partial data, under the control of the controlling unit 18 (step S208). After that, the selecting unit 17a judges whether the specifying unit 17c specified that the search target group “i” has the same image taking site and the same image taking cross-section as those of the first partial data (step S209).

In this situation, if the specifying unit 17c specified that the search target group “i” has the same image taking site and the same image taking cross-section as those of the first partial data (step S209: Yes), the selecting unit 17a confirms the search target group “i” to be second partial data (step S210).

In this situation, after the process at step S210 is performed, or if “dT(i)” is equal to or larger than the threshold value “α” (step S207: No), or if the specifying unit 17c did not specify that the search target group “i” has the same image taking site and the same image taking cross-section as those of the first partial data (step S209: No), the selecting unit 17a judges whether “i=N” is satisfied (step S211). In this situation, if “i” is smaller than “N” (step S211: No), the selecting unit 17a sets “i=i+1” (step S212) and returns to step S206 where the selecting unit 17a calculates the absolute value “dT(i)” of the difference between the heart beat period of the next search target group “i” and the reference heart beat period.

On the contrary, if “i=N” is satisfied (step S211: Yes), the ultrasound diagnosis apparatus according to the second embodiment ends the selecting process. It is acceptable to configure the second embodiment so that the display process at step S208 is not performed.

As explained above, in the second embodiment, the specifying unit 17c specifies the group of ultrasound images having the same image taking site as that of the first partial data, out of the second partial data. Further, the specifying unit 17c specifies the group of ultrasound images having the same image taking cross-section as that of the first partial data, out of the second partial data. Further, when the specifying unit 17c has specified the group of ultrasound images having the same image taking site and the same image taking cross-section as those of the first partial data out of the second partial data, the obtaining unit 17b determines the specified group of ultrasound images to be the target from which the difference information is obtained.

In other words, according to the second embodiment, it is also possible to automatically judge whether the observation site is the same, which is judged by the operator in the first embodiment. Consequently, in the second embodiment, it is possible to further reduce the burden on the operator.

Further, it is also acceptable to configure the specifying unit 17c so as to perform modification examples described below. In one example, it is acceptable to configure the specifying unit 17c to specify a group of ultrasound images having the same image taking site out of the second partial data, based on body marks appended to the ultrasound images. In another example, in addition to the image taking site and the image taking cross-section, it is acceptable to configure the specifying unit 17c to specify ultrasound images having the same image quality as that of the first partial data out of the second partial data, by further calculating a parameter with which it is possible to evaluate the image quality such as a contrast value of each of the ultrasound images.

In yet another example, another arrangement is acceptable in which the specifying process performed by the specifying unit 17c is performed while using the second data group as the target. In that situation, when the specifying unit 17c has specified the group of ultrasound images having the same image taking site and the same image taking cross-section as those of the first partial data out of the second data group, the selecting unit 17a determines the specified group of ultrasound images to be a target from which the second partial data is selected.

In yet another example of the second embodiment, it is acceptable to configure the specifying unit 17c so as to specify the group of ultrasound images having the same image taking site as that of the first partial data, either out of the second data group or out of the second partial data. When the specifying unit 17c has specified the group of ultrasound images having the same image taking site as that of the first partial data out of the second data group, the selecting unit 17a determines the specified group of ultrasound images to be the target from which the second partial data is selected. Alternatively, when the specifying unit 17c has specified the group of ultrasound images having the same image taking site as that of the first partial data out of the second partial data, the obtaining unit 17b determines the specified group of ultrasound images to be the target from which the difference information is obtained.

Further, it is acceptable to configure the second embodiment in such a manner that the specifying unit 17c specifies a group of ultrasound images having the same image quality as that of the first partial data out of the second data group and that the selecting unit 17a determines the specified group of ultrasound images to be the target from which the second partial data is selected.

According to these modification examples also, it is possible to further reduce the burden on the operator.

In the first and the second embodiments, the examples are explained in which the process to select the second partial data is performed, after the ultrasound images are acquired. In a third embodiment, an example in which the process to select second partial data is performed during an acquisition of ultrasound images will be explained.

The image processing unit 17 according to the third embodiment is configured to be similar to the image processing unit 17 according to the first embodiment shown in FIG. 1; however, processes performed by the selecting unit 17a are different from those in the first embodiment. The third embodiment will be explained below, while a focus is placed on this difference.

In the third embodiment also, in the same manner as explained in the first embodiment, the selecting unit 17a calculates the reference heart beat period (T) based on the first partial data selected by the operator. It is also acceptable to configure the third embodiment so that the selecting process is performed by using a heart rate.

Further, the selecting unit 17a according to the third embodiment determines a group of ultrasound images acquired in a time frame different from that of the first data group to be a second data group, further selects second partial data, based on a detection result of a heart rate of the subject P detected during the acquisition of the second data group, and stores the selected second partial data into the image memory 15. In the third embodiment also, it is acceptable to configure the selecting unit 17a so as to select a group of ultrasound images of which the acquisition period is substantially equal to the acquisition period of the first partial data, as second partial data.

For example, in the third embodiment, prior to the image acquiring process, the operator adjusts the position of the ultrasound probe 1, so that the image taking site and the image taking cross-section become the same as those of the first partial data. Further, in the third embodiment, the operator adjusts the image quality so that the image quality becomes the same as that of the first partial data.

After the adjustments are made as described above, for example, a process to acquire images of the apical four chambers is started, so that a process performed by the selecting unit 17a according to the third embodiment is started. For example, the operator causes the selecting unit 17a according to the third embodiment to start performing the process, by pressing a switch for a stored-data acquiring mode included in the input device 3. First, every time a group of ultrasound images corresponding to one heart beat is acquired, the selecting unit 17a detects the heart rate in the acquisition period of the group of ultrasound images, based on an electrocardiogram obtained from the electrocardiographic equipment 4. Further, like in the first embodiment, the selecting unit 17a judges whether the acquired group of ultrasound images corresponding to the one heart beat is the second partial data, based on the heart beat period (T′) which is represented by the detected heart rate, the reference heart beat period (T), and the predetermined threshold value (α).

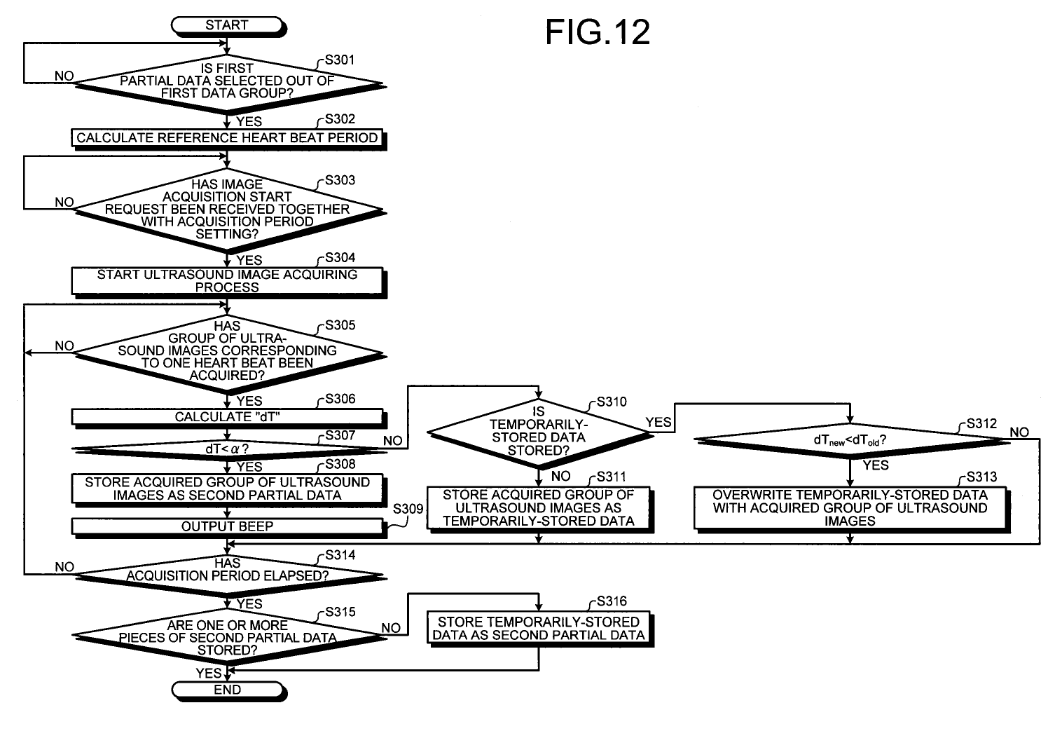

After that, the selecting unit 17a according to the third embodiment stores the group of ultrasound images selected as the second partial data, into the image memory 15. FIG. 11 is a drawing for explaining the selecting unit according to the third embodiment.

For example, the selecting unit 17a stores the group of ultrasound images selected as the second partial data into a second partial data storage area 15a (see FIG. 11) provided in the image memory 15.

After that, the controlling unit 18 according to the third embodiment exercises control so that the speaker of the output device 2 outputs information used for informing the operator of whether the selecting unit 17a has performed the storing process into the image memory 15. For example, if the selecting unit 17a has stored the second partial data, the controlling unit 18 causes the speaker of the output device 2 to output a beep. Another arrangement is also acceptable in which, if the selecting unit 17a has stored the second partial data, the controlling unit 18 causes the monitor of the output device 2 to display a character string indicating “NOW STORING DATA”.

The acquisition period of the second data group is set by the operator via the input device 3. In other words, the input device 3 receives the acquisition period of the second data group, so that the selecting unit 17a performs the process to select the second partial data during the acquisition period received by the input device 3. Another arrangement is also acceptable in which the selecting unit 17a calculates a remaining period of the acquisition period, so that the controlling unit 18 causes the monitor to display the remaining period calculated by the selecting unit 17a.

Also, another arrangement is acceptable in which the acquisition period of the second data group ends at the point in time when, for example, the operator presses a “freeze button” included in the input device 3.

In this situation, if no second partial data was selected, the selecting unit 17a stores a group of ultrasound images having the closest heart rate to the reference heart rate into the second partial data storage area 15a, as second partial data. In the third embodiment, if no second partial data was selected, the selecting unit 17a stores a group of ultrasound images having the closest heart beat period to the reference heart beat period into the second partial data storage area 15a, as second partial data. As shown in FIG. 11, to perform this process in the third embodiment, a temporarily-stored data storage area 15b, for example, is provided in the image memory 15.

In other words, after the selecting process is started, the selecting unit 17a temporarily stores the group of ultrasound images that was not initially selected as second partial data into the temporarily-stored data storage area 15b, instead of simply discarding the group of ultrasound images. After that, if a group of ultrasound images determined to be unselectable is newly found, the selecting unit 17a calculates the absolute value “dT_{new}” of the difference between the heart beat period of the group of ultrasound images and the reference heart beat period, as well as the absolute value “dT_{old}” of the difference between the heart beat period of the group of ultrasound images already stored in the temporarily-stored data storage area 15b and the reference heart beat period. If “dT_{new}” is smaller than “dT_{old}”, the selecting unit 17a overwrites the temporarily-stored data storage area 15b with the group of ultrasound images newly determined to be unselectable. On the contrary, if “dT_{new}” is equal to or larger than “dT_{old}”, the selecting unit 17a discards the group of ultrasound images newly determined to be unselectable.

The selecting unit 17a performs the process described above during the acquisition period. Further, if no second partial data is stored in the second partial data storage area 15a at the point in time when the acquisition period ends, the selecting unit 17a determines the temporarily-stored data stored in the temporarily-stored data storage area 15b to be second partial data. After that, the selecting unit 17a moves the temporarily-stored data to the second partial data storage area 15a.

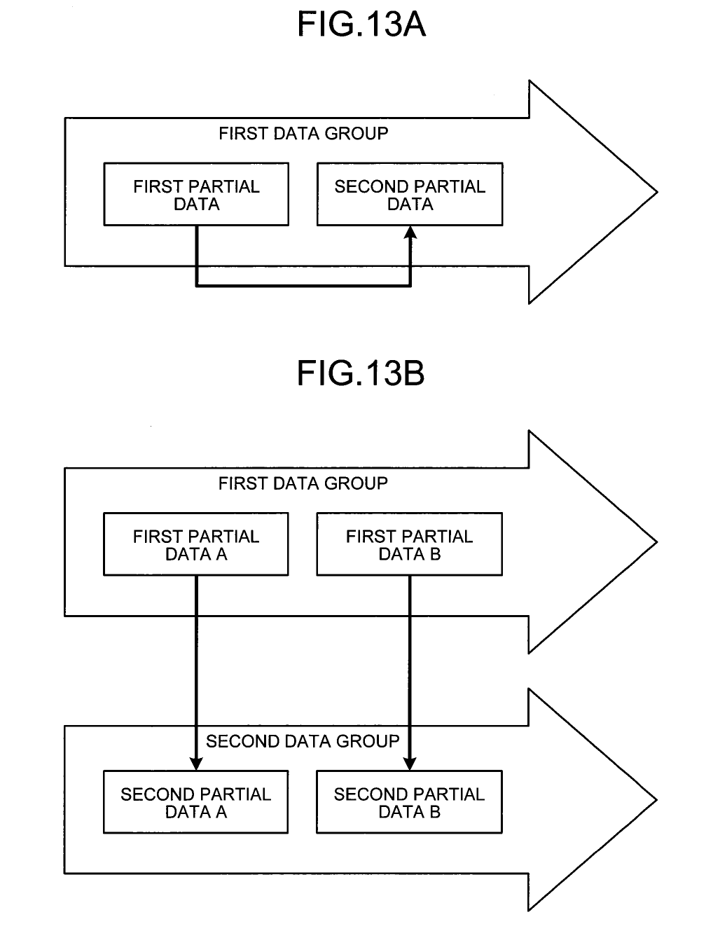

Next, a process performed by the ultrasound diagnosis apparatus according to the third embodiment will be explained, with reference to FIG. 12. FIG. 12 is a flowchart for explaining a selecting process performed by the ultrasound diagnosis apparatus according to the third embodiment. The analyzing process performed by the ultrasound diagnosis apparatus according to the third embodiment is the same as the analyzing process performed by the ultrasound diagnosis apparatus according to the first embodiment explained with reference to FIG. 7. The explanation thereof will be therefore omitted.

As shown in FIG. 12, the ultrasound diagnosis apparatus according to the third embodiment judges whether first partial data is selected out of a first data group (step S301). In this situation, if no first partial data is selected (step S301: No), the ultrasound diagnosis apparatus according to the third embodiment goes into a standby state.

On the contrary, if first partial data is selected (step S301: Yes), the selecting unit 17a, which has been notified by the controlling unit 18 that the first partial data is selected, calculates a reference heart beat period (T) (step S302). Further, the selecting unit 17a judges whether an image acquisition start request has been received together with an acquisition period setting (step S303). In this situation, if no image acquisition start request has been received (step S303: No), the ultrasound diagnosis apparatus according to the third embodiment goes into a standby state.

On the contrary, if an image acquisition start request has been received (step S303: Yes), the ultrasound diagnosis apparatus according to the third embodiment starts an ultrasound image acquiring process (step S304). At the same time, the selecting unit 17a also starts measuring an elapsed time period since the start of the acquiring process.

After that, the selecting unit 17a judges whether ultrasound images corresponding to one heart beat have been acquired by, for example, detecting an R-wave in an electrocardiogram (step S305). In this situation, if ultrasound images corresponding to one heart beat have not been acquired (step S305: No), the selecting unit 17a stands by until ultrasound images corresponding to one heart beat have been acquired.

On the contrary, when ultrasound images corresponding to one heart beat have been acquired (step S305: Yes), the selecting unit 17a calculates the absolute value “dT” of the difference between the heart beat period of the acquired group of ultrasound images and the reference heart beat period (step S306). Further, the selecting unit 17a judges whether “dT” is smaller than the threshold value “α” (step S307).

In this situation, if “dT” is smaller than the threshold value “α” (step S307: Yes), the selecting unit 17a stores the acquired group of ultrasound images as second partial data, into the second partial data storage area 15a (step S308). After that, under the control of the controlling unit 18, the speaker of the output device 2 outputs a beep (step S309).

On the contrary, if “dT” is equal to or larger than the threshold value “α” (step S307: No), the selecting unit 17a judges whether the temporarily-stored data storage area 15b has any temporarily-stored data stored therein (step S310). In this situation, if the temporarily-stored data storage area 15b does not have any temporarily-stored data stored therein (step S310: No), the selecting unit 17a stores the acquired group of ultrasound images into the temporarily-stored data storage area 15b as temporarily-stored data (step S311).

On the contrary, if the temporarily-stored data storage area 15b has temporarily-stored data stored therein (step S310: Yes), the selecting unit 17a judges whether the absolute value “dT_{new}” of the difference between the heart beat period of the acquired group of ultrasound images and the reference heart beat period is smaller than the absolute value “dT_{old}” of the difference between the heart beat period of the temporarily-stored data that is already stored and the reference heart beat period (step S312).

In this situation, if “dT_{new}” is smaller than “dT_{old}” (step S312: Yes), the selecting unit 17a overwrites the temporarily-stored data storage area 15b with the acquired group of ultrasound images serving as temporarily-stored data (step S313).

Subsequently, after the process at step S309 is performed, or after the process at step S311 is performed, or after the process at step S313 is performed, or if “dT_{new}” is equal to or larger than “dT_{old}” (step S312: No), the selecting unit 17a judges whether the acquisition period has elapsed (step S314). In this situation, if the acquisition period has not elapsed (step S314: No), the selecting unit 17a returns to step S305 where the selecting unit 17a judges whether a group of ultrasound images corresponding to one heart beat has newly been acquired.

On the contrary, if the acquisition period has elapsed (step S314: Yes), the selecting unit 17a judges whether the second partial data storage area 15a has one or more pieces of second partial data stored therein (step S315). In this situation, if the second partial data storage area 15a does not have any second partial data stored therein (step S315: No), the selecting unit 17a stores the temporarily-stored data into the second partial data storage area 15a, as second partial data (step S316), and the process ends.

On the contrary, if the second partial data storage area 15a has one or more pieces of second partial data stored therein (step S315: Yes), the selecting unit 17a ends the process.

As explained above, according to the third embodiment, while using the group of ultrasound images acquired in the time frame different from that of the first data group as the second data group, the selecting unit 17a selects the second partial data based on the detection result of the heart rate of the subject P detected during the acquisition of the second data group and stores the selected second partial data into the second partial data storage area 15a within the image memory 15.

In other words, according to the third embodiment, the image acquiring process is performed after the operator makes the adjustments so that the image taking site, the image taking cross-section, and the image quality become the same as those of the first partial data. Thus, the selecting process by the selecting unit 17a is performed during the image acquiring process in a real-time manner. As a result, according to the third embodiment, it is possible to automatically select the second partial data having a substantially equal heart rate and having the same observation site and the same image quality. Consequently, it is possible to easily improve the reliability of the quantitative analysis performed on the changes in the cardiac function between the mutually-different time frames.

Further, in the third embodiment, the controlling unit 18 exercises control so that the output device 2 outputs the information used for informing the operator of whether the selecting unit 17a has performed the storing process into the second partial data storage area 15a. As a result, according to the third embodiment, the operator is able to easily recognize whether second partial data has been acquired in a real-time manner.

Further, in the third embodiment, the input device 3 receives the acquisition period of the second data group, whereas the selecting unit 17a performs the process to select the second partial data during the acquisition period received by the input device 3. Consequently, according to the third embodiment, the operator is able to arbitrarily designate the execution time period of the selecting unit 17a.

Further, in the third embodiment, if no second partial data was selected, the selecting unit 17a stores the group of ultrasound images having the closest heart rate (the closest heart beat period) to the reference heart rate (the reference heart beat period) into the second partial data storage area 15a, as the second partial data. In other words, in the third embodiment, not only the process to select the second partial data, but also the process to select the temporarily-stored data is performed. As a result, according to the third embodiment, it is possible to make available the data groups used for performing the quantitative analysis on the changes of the cardiac function.

It is acceptable to apply modification examples explained below to the first to the third embodiments described above. More specifically, it is acceptable to configure the controlling unit 18 to exercise control so that the monitor in the output device 2 displays at least one of the ultrasound images belonging to the first partial data and at least one of the ultrasound images belonging to the second partial data. For example, the controlling unit 18 arranges an ultrasound image corresponding to an R-wave in the first partial data and an ultrasound image corresponding to an R-wave in the second partial data to be displayed next to each other. In another example, the controlling unit 18 matches the cardiac phases of the first partial data and the second partial data and displays the images as a moving picture.

In yet another example, it is acceptable to configure the controlling unit 18 to exercise control so that the monitor displays not only the difference information, but also an index value calculated based on the first partial data and an index value calculated based on the second partial data. For example, it is acceptable to configure the controlling unit 18 to exercise control so that the monitor displays an index value from the first partial data and an index value from the second partial data, based on which the difference information was calculated.

Further, an arrangement is acceptable in which the first partial data is selected by the selecting unit 17a and the specifying unit 17c described above out of the first data group, by using, for example, information about the reference heart rate, the image taking site, the image taking cross-section, and the like designated by the operator.

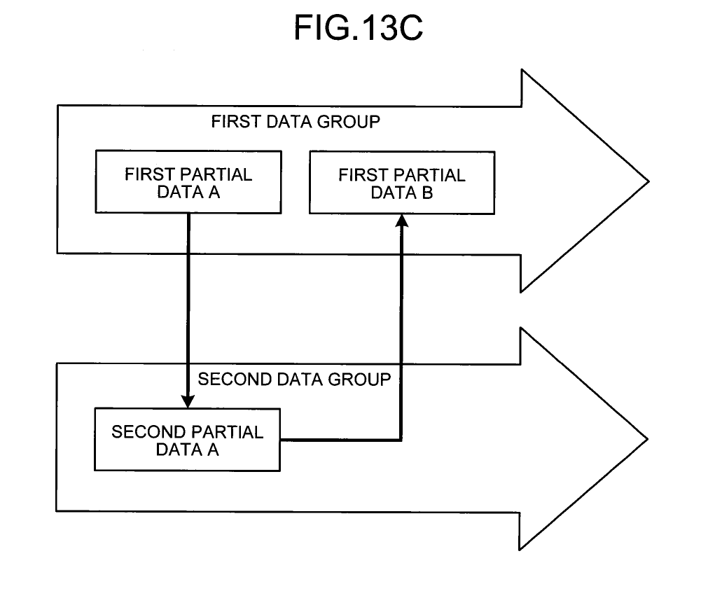

In yet another example, it is acceptable to configure the selecting unit 17a to perform any of the selecting processes shown in FIGS. 13A, 13B, and 13C. FIGS. 13A, 13B, and 13C are drawings for explaining modification examples of the selecting process. The data group serving as the target from which the second partial data is selected is represented by, as explained above, the group of ultrasound images of the subject P acquired in a time frame different from that of the first partial data. Thus, as shown in FIG. 13A, it is acceptable to configure the selecting unit 17a so as to select, out of the first data group, second partial data having a heart rate substantially equal to that of the first partial data selected out of the first data group.

In another example, if a plurality of pieces of first partial data have been selected out of the first data group, it is acceptable to configure the selecting unit 17a to select pieces of second partial data respectively corresponding to the plurality of pieces of first partial data, out of the second data group. For example, let us discuss a situation where, as shown in FIG. 13B, first partial data A and second partial data B have been selected out of the first data group. In that situation, as shown in FIG. 13B, the selecting unit 17a selects second partial data A having a heart rate substantially equal to that of the first partial data A and selects second partial data B having a heart rate substantially equal to that of the first partial data B, out of the second data group.

In yet another example, it is acceptable to configure the selecting unit 17a so as to, after having selected, out of the second data group, second partial data having a heart rate substantially equal to that of the first partial data selected out of the first data group, select again partial data having a heart rate substantially equal to that of the second partial data out of the first data group. For example, as shown in FIG. 13C, the selecting unit 17a selects second partial data A corresponding to first partial data A, out of the second data group.

It should be noted, however, that there is a possibility that one or more other pieces of partial data each having a heart rate close to the heart rate of the second partial data A may be present in the first data group, besides the first partial data A. When an analysis is performed by using the difference information, it is desirable to make a comparison by using pieces of partial data having closer heart rates. For this reason, the selecting unit 17a performs the process to select second partial data while using the second partial data A as first partial data and using the first data group as a second data group. As a result, as shown in FIG. 13C for example, the selecting unit 17a is able to select first partial data B having a heart rate closer to the heart rate of the second partial data A than the heart rate of the first partial data A is, out of the first data group.

The first to the third embodiments and the modification examples above are explained in the situation where the ultrasound diagnosis apparatus performs the process to select the first and the second partial data, as well as the process to obtain and display the index values and the difference information. The first to the third embodiments and the modification examples described above are, however, applicable to a situation where the ultrasound diagnosis apparatus performs the process to select the first and the second partial data groups, whereas a workstation or the like other than the ultrasound diagnosis apparatus performs the obtaining process and the display process.

Furthermore, another arrangement is acceptable in which the process to select the group of images performed by the ultrasound diagnosis apparatus according to any of the first to the third embodiments and the modification examples is performed by an image processing apparatus provided independently of the ultrasound diagnosis apparatus. More specifically, it is acceptable to configure an image processing apparatus having the display control function of the input device 3, the image processing unit 17, and the controlling unit 18 shown in FIG. 1 so as to perform the image group selecting process described above by receiving data groups received from the ultrasound diagnosis apparatus or from a Picture Archiving and Communication System (PACS) database or a database of an electronic medical record system.

In addition, the medical images serving as the target of the image group selecting process performed by the image processing apparatus described above may be ultrasound images, X-ray Computed Tomography (CT) images taken by an X-ray CT apparatus, X-ray images taken by an X-ray diagnosis apparatus, or Magnetic Resonance Imaging (MRI) images taken by an MRI apparatus.

Further, the constituent elements of the apparatuses illustrated in the drawings are based on the functional concepts thereof. Thus, it is not necessary to physically configure the elements as indicated in the drawings. In other words, the specific mode of distribution and integration of the apparatuses is not limited to the examples shown in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a Central Processing Unit (CPU) and an image processing computer program (hereinafter, “image processing program”) analyzed and executed by the CPU or may be realized as hardware using wired logic.

Further, it is possible to realize the image processing method explained above by causing a computer such as a personal computer or a workstation to execute the image processing program prepared in advance. It is possible to distribute the image processing program via a network such as the Internet. Another arrangement is also acceptable in which the image processing program is recorded on a computer-readable recording medium such as a hard disk, a flexible disk (FD), a Compact Disk Read-Only Memory (CD-ROM), a magneto-optical disk (MO), a Digital Versatile Disk (DVD), or the like, so that the image processing program is executed after being read by a computer from the recording medium.

As explained above, according to the first to the third embodiments and the modification examples, it is possible to easily improve the reliability of the quantitative analysis performed on the changes in the cardiac function between the mutually-different time frames.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
an input unit configured to receive a group of ultrasound images corresponding to at least one heart beat from among a group of ultrasound images corresponding to a plurality of heart beats of a subject, as first partial data; and
a selecting unit configured to, while using a heart rate of the subject during an acquisition period of the first partial data as a reference heart rate, select a group of ultrasound images having a heart rate within a predetermined range from the reference heart rate as second partial data, out of a group of ultrasound images of the subject acquired in a time frame different from that of the first partial data.

2. The ultrasound diagnosis apparatus according to claim 1, further comprising: a controlling unit configured to exercise control so that a predetermined display unit displays at least one ultrasound image belonging to the first partial data and at least one ultrasound image belonging to the second partial data.

3. The ultrasound diagnosis apparatus according to claim 1, further comprising: a controlling unit configured to exercise control so that a predetermined display unit displays an index value calculated based on the first partial data and an index value calculated based on the second partial data.

4. The ultrasound diagnosis apparatus according to claim 1, wherein the selecting unit selects a group of ultrasound images of which an acquisition period is substantially equal to the acquisition period of the first partial data, as the second partial data.

5. The ultrasound diagnosis apparatus according to claim 1, further comprising:
an obtaining unit configured to obtain chronological difference information between pieces of partial data, by using the first partial data and the second partial data; and
a controlling unit configured to exercise control so that a predetermined display unit displays the difference information obtained by the obtaining unit.

6. The ultrasound diagnosis apparatus according to claim 5, wherein the obtaining unit calculates pieces of quantitative information quantifying cardiac wall motion information in a region of interest of the subject, based on the first partial data and the second partial data, and obtains a chronological difference between the pieces of quantitative information calculated based on the first partial data and the second partial data, as the difference information.

7. The ultrasound diagnosis apparatus according to claim 1, wherein the selecting unit performs a process to select the second partial data out of a second data group represented by a group of ultrasound images of the subject already acquired in a time frame different from that of a first data group represented by a group of ultrasound images out of which the first partial data is selected.

8. The ultrasound diagnosis apparatus according to claim 7, further comprising: a controlling unit configured to exercise control so that a predetermined display unit displays the group of ultrasound images selected by the selecting unit as the second partial data, as second candidate partial data, wherein
if an operator, who refers to the second candidate partial data displayed by the predetermined display unit under the control of the controlling unit, determines the second candidate partial data as a comparison target to be compared with the first partial data, the selecting unit confirms the second candidate partial data to be the second partial data.

9. The ultrasound diagnosis apparatus according to claim 7, further comprising: a specifying unit configured to specify a group of ultrasound images having a same image taking site as that of the first partial data, either out of the second data group or out of the second partial data, wherein
if the specifying unit has specified the group of ultrasound images having the same image taking site as that of the first partial data out of the second data group, the selecting unit determines the specified group of ultrasound images to be a target from which the second partial data is to be selected, whereas
if the specifying unit has specified the group of ultrasound images having the same image taking site as that of the first partial data out of the second partial data, the obtaining unit determines the specified group of ultrasound images to be a target from which chronological difference information between the pieces of partial data is to be obtained.

10. The ultrasound diagnosis apparatus according to claim 9, wherein
the specifying unit further specifies a group of ultrasound images having a same image taking cross-section as that of the first partial data, either out of the second data group or out of the second partial data, and
if the specifying unit has specified a group of ultrasound images having both the same image taking site and the same image taking cross-section as those of the first partial data out of the second data group, the selecting unit determines the specified group of ultrasound images to be a target from which the second partial data is to be selected, whereas
if the specifying unit has specified the group of ultrasound images having both the same image taking site and the same image taking cross-section as those of the first partial data out of the second partial data, the obtaining unit determines the specified group of ultrasound images to be a target from which the difference information is to be obtained.

11. The ultrasound diagnosis apparatus according to claim 1, wherein
the selecting unit determines a group of ultrasound images of the subject acquired in a time frame different from that of a first data group represented by a group of ultrasound images out of which the first partial data is selected, to be a second data group, and
the selecting unit selects the second partial data based on a detection result of a heart rate of the subject detected during the acquisition of the second data group and stores the selected second partial data into a predetermined storage unit.

12. The ultrasound diagnosis apparatus according to claim 11, further comprising: a controlling unit configured to exercise control so that a predetermined output unit outputs information used for informing an operator of whether the selecting unit has performed the storing process into the predetermined storage unit.

13. The ultrasound diagnosis apparatus according to claim 11, wherein
the input unit receives an acquisition period of the second data group, and
the selecting unit performs a process to select the second partial data during the acquisition period received by the input unit.

14. The ultrasound diagnosis apparatus according to claim 11, wherein, if no second partial data was selected, the selecting unit stores a group of ultrasound images having a closest heart rate to the reference heart rate into the predetermined storage unit, as the second partial data.

15. An image processing apparatus comprising:
an input unit configured to receive a group of medical images corresponding to at least one heart beat from among a group of medical images corresponding to a plurality of heart beats of a subject, as first partial data; and
a selecting unit configured to, while using a heart rate of the subject during an acquisition period of the first partial data as a reference heart rate, select a group of medical images having a heart rate within a predetermined range from the reference heart rate as second partial data, out of a group of medical images of the subject acquired in a time frame different from that of the first partial data.

16. An image processing method comprising:
a process performed by an input unit to receive a group of medical images corresponding to at least one heart beat from among a group of medical images corresponding to a plurality of heart beats of a subject, as first partial data; and
a process performed by a selecting unit to, while using a heart rate of the subject during an acquisition period of the first partial data as a reference heart rate, select a group of medical images having a heart rate within a predetermined range from the reference heart rate as second partial data, out of a group of medical images of the subject acquired in a time frame different from that of the first partial data.
